(19) United States
(12) Patent Application Publication
(10) Pub. No.: US 2009/0022775 A1
(43) Pub. Date: Jan. 22, 2009

(54) POLYMER BACKBONE FOR PRODUCING ARTIFICIAL TISSUE

(75) Inventors: Simon Champ, Ludwigshafen (DE); Matthias Maase, Speyer (DE); Sascha Deck, Ludwigshafen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/162,012

(22) PCT Filed: Jan. 24, 2007
(86) PCT No.: PCT/EP2007/050707

§ 371 Date: Jul. 24, 2008

(30) Foreign Application Priority Data
Jan. 24, 2006 (EP) 06001474.3

(51) Int. Cl. A61L 27/60; B29C 39/00

(52) U.S. Cl. 424/423; 264/298

(57) ABSTRACT

The invention relates to polymer scaffolds suitable for producing artificial tissues, in particular polysaccharide scaffolds, to processes for their preparation, to their use for producing artificial tissues, and to artificial tissues produced on the basis of such polymer scaffolds.

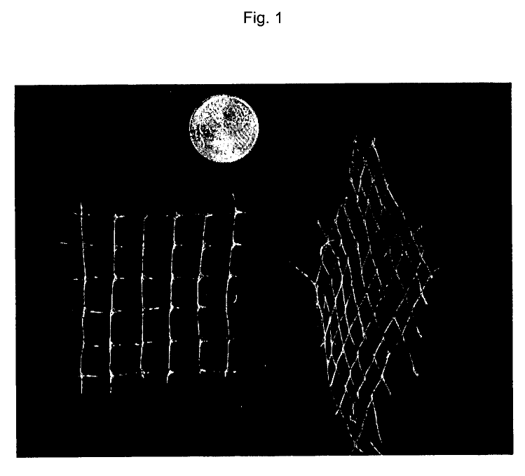

The invention relates to polymer scaffolds suitable for producing artificial tissues, in particular polysaccharide scaffolds, to processes for their preparation, to their use for producing artificial tissues, and to artificial tissues produced on the basis of such polymer scaffolds.

Whereas some lower vertebrates (e.g. newts) are able to repair extensive body and organ damage, in most mammals, including humans, the regeneration ability is low. Lesions caused in various ways (e.g. by injury, by pathogens or by autoimmune reactions) in most tissues and organs of humans and other mammals are not repaired after removal of the harmful factor by formation of new functional tissue, but are merely patched up by filling up with a particular connective tissue (scar formation), thus possibly affecting the further functionality of the particular organ. Even in those tissues which show a comparatively good regeneration ability (especially skin, connective tissues and their derivatives, e.g. bones), restoration of the original function is time-consuming and unpleasant for the patient, especially in the case of extensive lesions (e.g. large-area burns). Therapy therefore focuses on maintaining or minimally damaging modes of treatment, in which connection mention should be made of minimally invasive surgery (laparoscopy). However, even with maximal optimization of the medical and surgical treatment methods it is not possible to entirely preclude large-scale tissue damage caused iatrogenically or by the abovementioned factors. Possible means of restoring the function of damaged or removed organs are therefore sought.

Conventional approaches to this are transplantation and mechanical prosthetics, the latter being currently limited to parts of the body which are substantially biochemically and electrophysiologically inert (e.g. joints, lenses of the eye, heart valves). The only possible restorative treatment for tissues and organs having biochemical activity (e.g. heart, lung, liver, kidney) at present is transplantation. Although it leads in many cases to complete functional replacement, the known disadvantages are serious; mention should be made in this connection primarily of the shortage of donor organs, the need for life-long immunosuppression to avoid rejection reactions and the risk of transmission of pathogens, especially viruses. The idea of being able to restore functional tissues and possibly even whole organs is therefore of great therapeutic interest.

An approach which is currently receiving much attention is to employ pluripotent stem cells, which are able to differentiate to give various tissues, for regenerative purposes. The original assumption was that stem cells insert themselves into lesions (e.g. infarcted myocardial tissue) and of their own accord construct or regenerate functional tissue, as, after all, they do during natural ontogenesis. However, it is already clear that this is less trivial than assumed, and large-area lesions cannot be straightforwardly treated in this way. In particular, restoration of tissue with a complex structure, to say nothing of whole organs, is evidently not possible in this way.

As is now known, the presence of organizational signals is an essential factor for the growth of tissue or organs. During natural ontogenesis, these are mediated by a complex and only partly understood network of diffusible factors and cell-cell interactions (known as Spemann's inducers). In this connection, an essential role is played in regeneration processes in the complete organism by the arrangement of the cells in preformed structures, especially into the basic framework of the “extracellular matrix”. The “extracellular matrix” refers to the totality of all the structures which are separated from the cells but are always still in contact therewith. The extracellular matrix is important for the stability and functionality of many tissues, both as attachment point for the cells of the tissue and because of its own intrinsic properties, such as, for example, permeability and mechanical stability, and it is also important, owing to its specificity for particular cell types, for maintaining the organization of tissues (morphostasis) and homeostasis. Organization of tissues refers in this connection to the correct spatial arrangement of the cells, and homeostasis refers to their maintenance over time, even with changing stresses.

The removal, expansion and stimulation of living cells of various tissue types from a living organism, including that of a patient, are generally familiar to the skilled worker. However, the provision of synthetic preformed structures (matrices) into which cells can insert themselves and onto which cells can attach themselves in such a way that tissues which are capable of functioning and are suitable for reimplantation, ideally whole organs, can be formed still involves a substantial problem.

Colonization of preformed synthetic structures by living cells can in principle take place in vitro, but post-implantation processes are also possible, e.g. fusion of an artificial bone tissue with natural bone.

The use of artificial tissues or organs is, besides the use in regenerative therapy, as model in medical/pharmaceutical research, specifically in the area of drug targeting, an attractive alternative to the animal organs regularly used to date, because not only is it possible to work directly with human tissues, but there is also the possibility of better standardization and thus reproducibility.

The production of tissues and organs in vitro is frequently referred to as tissue engineering (see, for example, P. L. Pabst, “Tissue engineering: a historical review as seen through the US Patent Office”, Expert Opin. Ther. Patents 13 (2003): 347-352; L. G. Griffith and G. Vaughton, “Tissue Engineering—Current Challenges and Expanding Opportunities”, Science 295 (2002): 1009-1012; E. Pennisi, “Tending Tender Tendons”, Science 295 (2002): 1011).

Initial attempts to produce artificial tissue employed cartilage, a tissue which is rich in extracellular matrix and has little metabolic and biochemical activity and therefore regenerates poorly. U.S. Pat. No. 5,041,138 and U.S. Pat. No. 5,736,372 describe retention of the spatial shape of artificial tissue pieces even after degradation of the synthetically preformed structure, and the possibility of such artificial tissue pieces even being able to grow to the correct extent, which is important for example in pediatric use. It is therefore possible in this case to speak of complete restitution and not just of a prosthesis. However, U.S. Pat. No. 5,041,138 and U.S. Pat. No. 5,736,372 relate exclusively to the production of cartilaginous structures (e.g. ears, nose, esophagus) which, although having a complex shape macroscopically, exhibit only a slightly differentiated fine structure and low metabolic activity.

Various classes of organic polymers are suitable as material for such synthetic preformed structures. An essential aspect in this connection is that the substance must not induce any inflammation or rejection reaction, which rules out many of the versatile protein-like materials from the outset. In addition, the substance should be biodegradable, ideally at a rate corresponding to that of the replacement by biogenic structures in the particular organ. This should make it possible for the synthetic matrix to be replaced imperceptibly by natural tissues/structures while retaining the shape without critical phases of reduced mechanical stability. The degradation should preferably proceed not with swelling/disruption but by erosion, so that the mechanical stability of the synthetic structure is retained for as long as possible. In addition, no toxic monomers or oligomers should be formed in this degradation.

This makes certain demands both on the three-dimensional structure of the preformed synthetic matrix and on the material of which it is composed.

A general review of polymeric biomaterials is given for example by L. G. Griffith, “Polymeric Biomaterials”, Acta Mater. 48 (2000), 263-277. Mention is made both of natural materials such as collagen and fibrin, and of synthetic polymers such as polyglycolides and polylactide. For shaping, for example a polyglycolide is dipped in a polylactide solution in CHCl₃, and the wetted material is shaped in a mold. However, this way of producing three-dimensional scaffolds is not very precise, nor can it be applied to the production of scaffolds which are as small as desired or have a complex shape. In addition, the process is restricted to materials which have a relatively low softening point or melting point.

U.S. Pat. No. 5,328,603 describes a process for producing cellulose beads in the submillimeter range, which are intended to be employed in chromatographic methods. In this case, firstly cellulose is solubilized by chaotropic salts and subsequently the atomized solution is put into a medium which does not dissolve cellulose.

WO 03/029329 describes the production of a cellulose extrudate by solubilizing cellulose in an ionic liquid and subsequently extruding the solution into an aqueous medium. The production of two- or three-dimensional cellulose structures is not described.

The stochastic processes which were initially employed for three-dimensional shaping of biopolymers and which all essentially act by foaming of the polymer lead to inadequate results because, for physical reasons, a vesicular structure with numerous unconnected cavities which are separated from one another by polymeric material is achieved instead of the desired substantially continuous, preferably branched channel structure. Approaches based on casting molds by contrast lead, for practical reasons, to an exclusive channel structure which provides cells with possibilities for attachment and accumulation to only a limited extent. In addition, application thereof to materials which do not melt, like most polysaccharides, is difficult. The use of native biogenic polymers may lead to the formation of sponge-like structures in which channel and cavity shapes exist but which are mostly of limited mechanical stability. There is no possibility of differentiated structural configuration or doping with growth factors or signaling factors in any of the three cases mentioned. This also applies in particular to bacterial cellulose (A. Svensson et al., Biomaterials 26 (2005), 419-431) which therefore, despite its properties being otherwise favorable, is suitable only for tissue pieces which are spatially simple and simple in terms of basic structure, e.g. artificial joint cartilage.

In the precision engineering sector, an appropriate degree of control of the shaping process allows the miniaturization of processes from the CAD/CAM (computer aided design/computer aided manufacturing) sector, now frequently referred to as desktop manufacturing or rapid prototyping. In these processes, a three-dimensional model of the object to be manufactured is created in a computer and is then manufactured without further intermediate stages by an automated tool which is controlled by the computer. In these cases, the computer typically implements an algorithm which automatically breaks down the three-dimensional model into a number of finite elements suitable for implementation and completes them successively.

Build-up processes refer in this connection to those in which there is no cutting out of voids from an originally coherent block of material, but the material is loaded on stepwise during the shaping process. In this case, the intended cavities can either remain empty, i.e. be filled with a working medium (air or a liquid medium in which the material is insoluble), or be filled with a space-occupying substance which can be removed after completion of the shaping process for example by solvents or heating. There are various possible variations of build-up processes including chemical and/or photochemical reactions.

The implementations of the build-up processes can in principle be assigned to the two categories of printing (discontinuous) and plotting (continuous). In printing, the material is loaded on in screen or raster fashion, preferably from an array of nozzles, whereas an essentially uninterrupted material strand is extruded in the plotting. The plotting requires a greater expenditure of time, loading technology and control algorithms but leads to more uniform and predictable results. Both plotting and printing and their use in the tissue engineering sector are described in the prior art (e.g. V. L. Tsang and S. N. Bhatia, “Three-dimensional tissue fabrication”, Advanced Drug Delivery Reviews 56 (2004): 1635-1647; A. Pfister et al., “Biofunctional Rapid Prototyping for Tissue-Engineering Applications; 3D Bioplotting versus 3D Printing”, Journal of Polymer Science [Part A: Polymer Chemistry], Vol. 42 (2004), 624-638); E. Sachlos and J. T. Czernuszka, “Making Tissue Engineering Scaffolds Work”, Europ. Cells and Materials 5 (2003): 29-40; D. W. Hutmacher, “Scaffold design and fabrication technologies for engineering tissues”, J. Biomater. Sci. Polymer Edn. 12 (2001): 107-124). However, the polymers employed in said documents are only those having a lower softening point or melting point and are thus in fact extrudable.

It was an object of the present invention to provide a process making it possible to produce precisely two- and/or in particular three-dimensional structures which can be used as scaffold in the formation of tissues which are reimplantable or suitable as models in research, for example as scaffold in the field of tissue engineering. The process was intended in particular also to permit the use of scaffold materials which, although advantageous from the biological/medical viewpoint, cannot easily be worked because of their physicochemical properties such as melting point, moldability or solubility, with conventional processes for scaffold formation.

The object has been achieved by a process for producing two- or three-dimensional scaffolds of biodegradable and biocompatible polymers which comprises the following steps:
(i) solubilization of a biodegradable and biocompatible polymer in a chaotropic liquid; and
(ii-a) substantially continuous extrusion of the solution obtained in the first step into a liquid medium which is miscible with the chaotropic liquid but in which the polymer is substantially insoluble, by means of a needle, where the needle and the resulting scaffold move relative to one another in two or preferably three dimensions during the extrusion step; or
(ii-b) extrusion of the solution obtained in the first step into a liquid medium which is miscible with the chaotropic liquid but in which the polymer is substantially insoluble, by means of a needle to form individual straight, curved or bent polymer strands, where the needle and the resulting polymer strand move relative to one another during the extrusion step, if appropriate isolation of the polymer strands from the liquid medium and linkage of the polymer strands to form a two- or three-dimensional scaffold.

Any biodegradable and biocompatible polymer is suitable in principle as material.

A polymer is referred to as “biodegradable” in the context of the present invention when it can be degraded chemically or enzymatically, under the conditions prevailing in the organism, within a suitable period, e.g. within one year, preferably over the course of weeks or months, to monomers or oligomers which are soluble in body fluids.

A polymer is referred to as “biocompatible” in the context of the present invention if neither the polymer nor its monomeric or oligomeric degradation products exert a harmful, e.g. toxic and/or proinflammatory effect on the organism, and in particular if the degradation products can either be excreted as such or after transformation customary in the organism (cleavage, coupling etc) and/or be utilized in metabolism, without a toxic, immunological (e.g. proinflammatory), mutagenic, carcinogenic, cocarcinogenic or morphogenic (e.g. teratogenic) effect occurring.

A review of suitable polymers is given for example by Toshio Hayashi, “Biodegradable Polymers for Biomedical Uses”, Prog. Polym. Sci., Vol. 19, 663-702, (1994).

The term “scaffold” means in the context of the present invention a spatial structure which comprises at least two straight, curved and/or bent rods or strands, with at least one strand or rod overlap or contact usually being present. Overlap means in this connection that the angle between the strands is not equal to 0, whereas contact also includes the angle 0 (e.g. when strands lie parallel to one another). It is also possible for non-rodlike, e.g. flat, spiral or circular elements to be included in the scaffold.

“Rod” or “strand” means a structure which in the extended state (“straight rod/strand”) is substantially linear, i.e. a spatial shape which extends in one dimension. A “strand” is a rod obtainable by extrusion. Bent or curved rods/strands have, considered as complete structure, an extent in two dimensions.

In the context of the present invention, a structure fills up a particular dimension if its extent in this dimension amounts to more than one, preferably more than two, strand or rod diameters. Thus, “beads” according to U.S. Pat. No. 5,328,603 are zero-dimensional and extended single strands are one-dimensional. A “three-dimensional scaffold” is a scaffold which fills up three dimensions. A “two-dimensional scaffold” has an extent in two dimensions. Although individual curved or bent strands are also two-dimensional according to the above definition, in the context of the present invention a two-dimensional scaffold is intended to mean one which includes at least two rods/strands which overlap or are in contact in at least one point and whose joint extent is restricted to two dimensions. Multidimensional structures include, besides the scaffolds of the invention, also shapes without strand overlaps, such as, for example, loops or coils. Although these do not correspond to the term “scaffold” used in the context of the present invention, they may form part of the scaffolds of the invention.

The term “solubilization” refers in the context of the present invention to a conversion, which can be achieved without substantial heating, of the scaffold material (polymer) into a flowable, pourable or extrudable state. This entails the polymer being converted into a solvated state in which, however, the individual polymer molecules need not be completely enveloped by a solvation sheath. It is essential for the polymer to be converted by the solubilization into a liquid state or at least a softening state. The term “without substantial heating” means employing, for the solubilization, temperatures not exceeding 200° C., preferably not exceeding 150° C., particularly preferably not exceeding 120° C. and in particular not exceeding 100° C.

Substances are referred to as “chaotropic” if they are able to disrupt supermolecular associations of macromolecules by disturbing or influencing the intermolecular interactions without at the same time influencing the intramolecular covalent bonds.

The term “extrusion” is in the context of the present application not confined to a particular fabrication technique but refers very generally to the substantially continuous forcing of a flowable material out through a relatively narrow aperture (i.e. a nozzle in the widest sense), e.g. through a needle. “Substantially continuous” means in this connection that the extrusion operation can also be interrupted repeatedly, e.g. to produce individual polymer strands (for example as in step (ii-b)) or to change to a different spatial plane in step (ii-a). However, it does not take place with periodic interruptions such that only zero-dimensional structures such as, for example, beads are produced.

A “substantially continuous” polymer rod is a polymer structure which is one-dimensional in the extended state, i.e. a structure which is not produced by joining together and/or fusing zero-dimensional structures in a particular arrangement. It is preferred in this connection for the rod to be of substantially uniform thickness, especially if it shows no rhythmic alternation of thicker and thinner segments, and if the molecular structure is substantially uniform in the dimension direction, in particular if the molecular structure shows no rhythmic alternation in the dimension direction. It is further preferred for the polymer chains in the rod to be aligned substantially parallel to one another and to the longitudinal direction of the rod, especially if polymer chains lying parallel overlap in the longitudinal dimensions so that areas of contact between the molecules are produced. It is particularly preferred in this connection for the extent of the overlap to be substantially uniform over the entire length of the rod. In a preferred embodiment of the process, the overlapping of the polymer chains leads to the formation of partially crystalline regions. In a further embodiment, subsequent stabilization by covalent crosslinking in the region of overlap is possible. The expression “substantially” means that usual deviations caused for example by the extrusion step are tolerated.

The term “needle” refers to any type of nozzle through which the solution produced in the first step can be forced continuously.

The “movement of needle and scaffold or polymer strand relative to one another” means that during the extrusion step (ii) either the needle alone and the scaffold or the polymer strand or the container which comprises the liquid medium into which the polymer is extruded, or both, can move. The movement takes place considered over the whole of step (ii-a) in two spatial directions (two-dimensional movement) or preferably in all three spatial directions (three-dimensional movement), or considered over the whole of step (ii-b) in one or two spatial directions. Movement in one spatial direction results in straight polymer strands, whereas a two-dimensional relative movement leads to curved or bent strands. It is also possible in step (ii-b) for there to be a three-dimensional relative movement of needle and polymer strand, e.g. to form spiral or circular elements which may also be part of the scaffold but are preferably incorporated to only a minor extent.

“Three-dimensional movement” or “movement in three spatial directions” means that the position of the needle orifice can be varied in all three spatial dimensions relative to the scaffold which has so far been formed. In a particular embodiment of step (ii-a), the extrusion mechanism can be displaced in all three spatial dimensions. In an alternative embodiment of step (ii-a), the extrusion mechanism can be shifted in at least two spatial dimensions, and the scaffold which has so far been formed can be shifted in at least one spatial dimension, so that the missing spatial dimensions (degrees of freedom) of the extrusion mechanism can be made up by displacing the scaffold which has so far been formed. In a further alternative embodiment of step (ii-a), the extrusion mechanism can be shifted in at least one dimension, and the scaffold which has so far been formed in at least two dimensions, so that the missing degrees of freedom of the extrusion mechanism are made up by the mobility of the scaffold which has so far been formed. In a further alternative embodiment of step (ii-a), the extrusion mechanism is substantially immovable, while the scaffold which has so far been formed can be shifted in all three spatial dimensions.

An analogous statement applies to the “one- or two-dimensional movement” in step (ii-a) or (ii-b), i.e. either the extrusion mechanism or the resulting polymer strand (or more accurately the container into which the latter is extruded) is movable. In the case of two-dimensional relative movement, it is also possible for the extrusion mechanism to move in one spatial direction and the polymer strand in a spatial direction different therefrom.

“Substantially insoluble” means that the polymer has a solubility of less than 5 g/l, preferably less than 0.5 g/l and particularly preferably less than 0.05 g/l, in the liquid medium.

A “liquid medium” refers to a medium whose physicochemical properties are mainly determined by those of a liquid solvent. The liquid medium may also have a gelatinous consistency as a result of the presence of soluble or swellable macromolecules.

“Alkyl” stands for a linear or branched alkyl radical. Alkyl is preferably C₁-C₆-alkyl. C₁-C₆-alkyl stands for a linear or branched alkyl radical having 1 to 6 carbon atoms. Examples thereof are methyl, ethyl, propyl, isopropyl, n-butyl, sec-butyl, isobutyl, tert-butyl, n-pentyl, n-hexyl and constitutional isomers thereof. C₁-C₄-Alkyl stands for a linear or branched alkyl radical having 1 to 4 carbon atoms. Examples thereof are methyl, ethyl, propyl, isopropyl, n-butyl, sec-butyl, isobutyl and tert-butyl.

C₁-C₆-Alkoxy stands for a C₁-C₆-alkyl radical linked via an oxygen atom. Examples thereof are methoxy, ethoxy, propoxy, isopropoxy, n-butoxy, sec-butoxy, isobutoxy, tert-butoxy, pentoxy, hexoxy and constitutional isomers thereof. C₁-C₄-Alkoxy stands for a C₁-C₄-alkyl radical linked via an oxygen atom. Examples thereof are methoxy, ethoxy, propoxy, isopropoxy, n-butoxy, sec-butoxy, isobutoxy and tert-butoxy.

C₁-C₆-Alkoxy-C₁-C₆-alkyl stands for a C₁-C₆-alkyl radical in which one or more hydrogen atoms are replaced by a C₁-C₆-alkoxy radical. Examples thereof are methoxymethyl, ethoxymethyl, propoxymethyl, 1- and 2-methoxyethyl, 1- and 2-ethoxyethyl, 1- and 2-propoxyethyl and the like. C₁-C₄-Alkoxy-C₁-C₄-alkyl stands for a C₁-C₄-alkyl radical in which one or more hydrogen atoms are replaced by a C₁-C₄-alkoxy radical. Examples thereof are the abovementioned radicals.

Aryl stands for a carboaromatic radical preferably having 6 to 14 carbon atoms. Examples thereof are optionally substituted phenyl, optionally substituted naphthyl, optionally substituted anthracenyl and optionally substituted phenanthrenyl. Examples of suitable substituents are halogen, C₁-C₆-alkyl, NO₂, OH and CN. Aryl is preferably phenyl or substituted phenyl such as tolyl, xylyl, nitrophenyl or chlorophenyl.

Aryl-C₁-C₆-alkyl stands for an aryl radical linked via C₁-C₆-alkyl, preferably C₁-C₂-alkyl, such as benzyl or 2-phenylethyl.

Aryloxy stands for an aryl radical linked via oxygen, such as phenoxy.

Aryl-C₁-C₆-alkoxy stands for a C₁-C₆-alkoxy radical, preferably C₁-C₂-alkoxy radical, in which one hydrogen atom is replaced by an aryl group, e.g. benzoxy.

Aryloxy-C₁-C₆-alkyl stands for a C₁-C₆-alkyl radical, preferably C₁-C₂-alkyl radical, in which one hydrogen atom is replaced by an aryloxy group.

Halogen stands for fluorine, chlorine, bromine or iodine, in particular for fluorine or chlorine.

Acid anions of C₁-C₆ monocarboxylic acids are the acid anions of aliphatic C₁-C₆ monocarboxylic acids. Examples thereof are acetate, propionate, butyrate, isobutyrate, pentanoate, hexanoate and the like.

Monoanions and dianions of C₂-C₆ dicarboxylic acids are the monovalent anions or the dianions of aliphatic C₂-C₆ dicarboxylic acids, e.g. the monoanions or dianions of oxalic acid, malonic acid, succinic acid, adipic acid and the like.

The statements made hereinafter about the preferred embodiments of the subject-matters of the invention apply both taken on their own and in combination with one another.

In a preferred embodiment of the process of the invention, the polymeric scaffold material is an organic polymer. An organic polymer means in this connection a polymer whose monomers are essentially organic molecules, e.g. alcohols, especially dialcohols and polyalcohols, carboxylic acids, especially hydroxy dicarboxylic acids and amino acids, amines, especially diamines and polyamines, and amino acids, and saccharides, especially glucose and fructose units. “Essentially organic molecules” means that these may also comprise inorganic components, e.g. metal cations or halide ions, but the overall nature of the molecule is organic.

In a particularly preferred embodiment, the polymer is a biopolymer. A biopolymer means in this connection a polymer whose monomers occur in nature, e.g. saccharides and amino acids, and especially a polymer whose complete structure occurs in nature. Examples of biopolymers are proteins, e.g. silk protein, and polysaccharides, e.g. cellulose, cellulose derivatives, chitin, chitosan, dextran, hyaluronic acid, chondroitin sulfate, xylan and starch.

The polymer is more preferably selected from polysaccharides and modified polysaccharides and in particular from polysaccharides. These not only satisfy the requirements in chemical and mechanical terms made in the field of tissue engineering for suitable materials; they are additionally, in contrast to many proteins, immunologically acceptable. Examples of suitable polysaccharides are cellulose, cellulose derivatives, chitin, chitosan, dextran, hyaluronic acid, chondroitin sulfate, xylan and starch.

In an even more preferred embodiment, cellulose or a cellulose derivative is employed in the process of the invention. Examples of suitable cellulose derivatives are methylcellulose, ethylcellulose, propylcellulose, hydroxyethylcellulose and hydroxypropylcellulose. Cellulose is used in particular. Any known form of cellulose can be employed as cellulose, e.g. from pulp, cotton, cellulose obtained from paper or bacterial cellulose.

The polymer is suitably subjected to mechanical size reduction, e.g. by grinding and/or shredding, before the solubilization.

The polymer can be employed in step (i) as such or together with further components. Preferred additional components are those which advantageously influence the construction of the scaffold and/or the subsequent use of the scaffold. Examples of suitable components are inorganic particles, e.g. hydroxyapatite particles and non-structural biopolymers, i.e. biopolymers different from the scaffold polymer, e.g. proteins, protein fragments, peptides or certain carbohydrates. In a preferred embodiment, non-structural biopolymers are used as additional components which favor the adhesion of cells and/or the formation of organized supercellular structures. Examples of suitable non-structural biopolymers are matrix proteins, e.g. fibronectin, vitronectin, collagen, laminin, lectins, tissue extracts, growth factors, e.g. VEGF, or fusion proteins or other derivatives of said proteins. Further suitable biopolymers are proteins or peptides which comprise the amino acid motif R-G-D, also adhesion-favoring carbohydrates such as sialyl-Lewis^{x} or fragments thereof or carbohydrates which are bioactive in other ways, such as heparin or fragments thereof. The corresponding molecules may in each case be linked covalently or non-covalently to polymer molecules.

When the polymer employed in step (i) comprises one or more of said biopolymers, the latter are present in an amount of, preferably, 0.1% by weight to 5% by weight, in particular from 1% to 2% by weight, based on the total weight of the scaffold polymer.

When the scaffold polymer comprises inorganic particles such as hydroxyapatite, these are present in an amount of, preferably, 1 to 20% by weight, in particular from 5 to 10% by weight, based on the weight of the scaffold polymer.

In a preferred embodiment of the invention, the chaotropic liquid is substantially anhydrous. “Substantially anhydrous” means that the chaotropic liquid comprises less than 5% by weight of water, preferably less than 2% by weight of water, particularly preferably less than 1% by weight of water, based on the total weight of the chaotropic liquid.

In a preferred embodiment of the invention, the chaotropic liquid is substantially free of nitrogen-containing bases. “Substantially free of nitrogen-containing bases” means that the chaotropic liquid comprises less than 5% by weight, preferably less than 2% by weight, particularly preferably less than 1% by weight, of nitrogen-containing bases, based on the total weight of the chaotropic liquid. Nitrogen-containing bases are for example ammonia, amines and aromatic or nonaromatic heterocycles having at least one basic nitrogen atom as ring member.

The chaotropic liquid is preferably liquid at a temperature not exceeding 150° C., e.g. in the temperature range from −100° C. to +150° C. or from 0 to +150° C. or from 50 to +150° C., particularly preferably not exceeding 120° C., e.g. in the temperature range from −50° C. to +120° C. or from 0 to +120° C. or from 50 to +120° C., and in particular not exceeding 100° C., e.g. in the temperature range from −10° C. to +100° C. or from 0 to +100° C. or from 50 to +100° C. This means that the chaotropic liquid has a melting point which preferably does not exceed 150° C., particularly preferably does not exceed 120° C. and in particular does not exceed 100° C.

The solubilization step can also be assisted by ultrasound.

In a specific embodiment of the invention, the heating takes place by microwave irradiation.

The solubilization preferably takes place at temperatures not exceeding 200° C., e.g. from 0 to 200° C. or preferably from 20° to 200° C. or particularly preferably from 50 to 200° C. or in particular from 100 to 200° C., particularly preferably not exceeding 150° C., e.g. from 0° C. to +150° C. or preferably from 20 to 150° C. or particularly preferably from 50 to 150° C. or in particular from 100 to 150° C., more preferably not exceeding 120° C., e.g. from 0° C. to 120° C. or preferably from 20 to 120° C. or particularly preferably from 50 to 120° C. or more preferably from 80 to 120° C. or in particular from 100 to 120° C. and in particular not exceeding 100° C., e.g. from 0° C. to +100° C. or preferably from 20 to 100° C. or particularly preferably from 50 to 100° C. or in particular from 80 to 100° C.

In a preferred embodiment of the invention, the chaotropic liquid is selected from liquid salts. Liquid salts are also referred to as ionic liquids. Ionic liquids generally mean salts in which the ions are only weakly coordinated so that these salts are liquid at relatively low temperatures, e.g. below 150° C. or below 100° C. or even at room temperature. In this case, the charge in at least one of the ions is delocalized, and at least one of the ions is organic in nature, thus preventing the formation of stable crystal lattices.

The liquid salt preferably has the formula Het⁺A^{x-}_{1/x}.

In this connection, Het⁺ is a positively charged N-alkylated, N-arylated, N-arylalkylated, N-alkoxylated, N-aryloxylated, N-arylalkoxylated, N-alkoxyalkylated and/or N-aryloxyalkylated nitrogen-containing heterocycle. In other words, Het⁺ is a positively charged nitrogen-containing heterocycle in which formally a ring nitrogen atom carries an alkyl radical, aryl radical, arylalkyl radical, alkoxy radical, aryloxy radical, arylalkoxy radical, alkoxyalkyl radical and/or an aryloxyalkyl radical bonded via its free electron pair, so that a positive charge results in the heterocycle, i.e. the positive charge of the heterocycle is attributable to substitution on the free electron pair of a ring nitrogen atom.

Alkyl in said radicals is preferably C₁-C₆-alkyl. Alkoxy in said radicals is preferably C₁-C₆-alkoxy. Aryl in said radicals is preferably phenyl. Arylalkyl in said radicals is preferably aryl-C₁-C₆-alkyl, such as benzyl or phenylethyl. Aryloxy in said radicals is preferably a phenyl radical linked via oxygen, e.g. phenoxy. Arylalkoxy in said radicals is preferably an aryl-C₁-C₆-alkoxy radical, e.g. benzoxy. Alkoxyalkyl in said radicals is preferably a C₁-C₆-alkoxy-C₁-C₆-alkyl radical. Aryloxyalkyl in said radicals is preferably an aryloxy-C₁-C₆-alkyl radical, in particular a phenyloxy-C₁-C₆-alkyl radical.

Depending on whether Het⁺ is an aromatic heterocycle or an alicyclic heterocycle in which the ring nitrogen atom is not part of a double bond, the nitrogen atom which formally produces the positive charge is substituted either once or twice by the abovementioned radicals.

A^{x-}_{1/x} is an anion in which x is 1, 2 or 3.

Het⁺ is preferably selected from
positively charged 5- or 6-membered aromatic heterocycles which comprise as ring member a group NR^{a} and optionally one or two heteroatoms or heteroatom-containing groups which are selected from N, O, S, NR^{b}, SO and SO₂,
positively charged 5- or 6-membered aromatic heterocycles which comprise as ring member a group NR^{a} and optionally one or two heteroatoms or heteroatom-containing groups which are selected from N, O, S, NR^{b}, SO and SO₂, and which are fused to a benzene ring, and
positively charged 5- or 6-membered saturated alicyclic heterocycles which comprise as ring member a group NR^{a}R^{a′} and optionally one or two heteroatoms or heteroatom-containing groups which are selected from O, S, NR^{b}, SO and SO₂,
   in which
R^{a} and R^{a′} are independently of one another C₁-C₆-alkyl, aryl, C₁-C₆-alkoxy, aryloxy, C₁-C₆-alkoxy-C₁-C₆-alkyl or aryloxy-C₁-C₆-alkyl and preferably C₁-C₆-alkyl or C₁-C₆-alkoxy-C₁-C₆-alkyl; and
R^{b} is hydrogen, C₁-C₆-alkyl, aryl, C₁-C₆-alkoxy, aryloxy, C₁-C₆-alkoxy-C₁-C₆-alkyl or aryloxy-C₁-C₆-alkyl and preferably C₁-C₆-alkyl or C₁-C₆-alkoxy-C₁-C₆-alkyl;
   where the alicyclic or aromatic heterocycles or the benzene rings to which the latter may be fused may have 1 to 5 substituents selected from C₁-C₆-alkyl, C₁-C₆-alkoxy and C₁-C₆-alkoxy-C₁-C₆-alkyl.

Het⁺ is particularly preferably selected from compounds of the formulae Het. 1 to Het. 15:

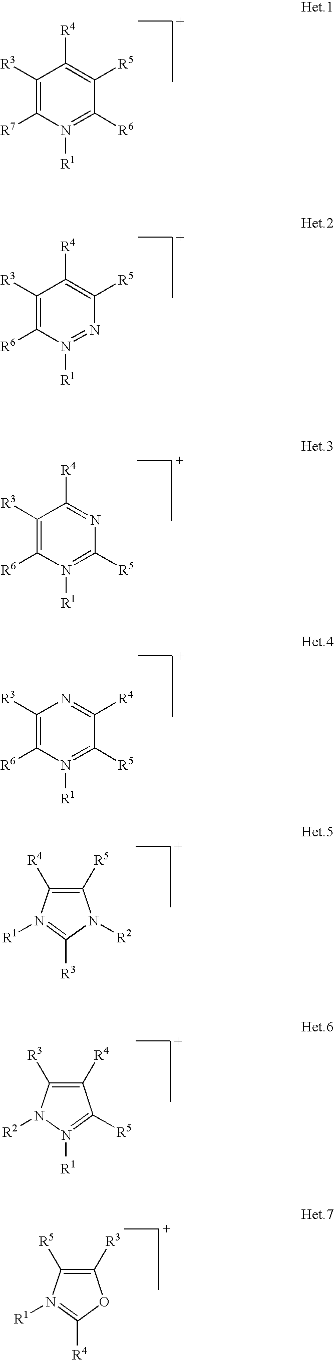

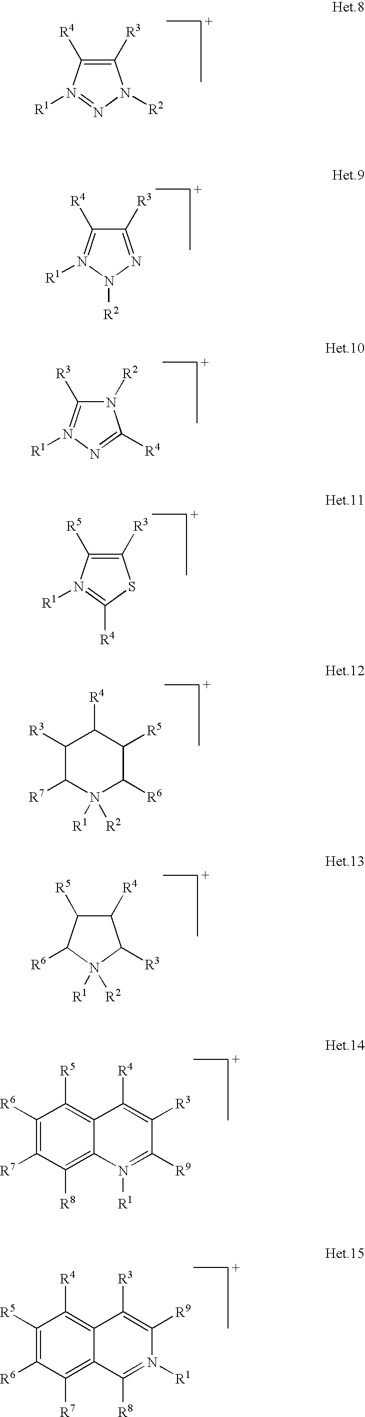

in which
R¹ and R² are independently of one another C₁-C₆-alkyl or C₁-C₆-alkoxy-C₁-C₆-alkyl; and
R³ to R⁹ are independently of one another hydrogen, C₁-C₆-alkyl, C₁-C₆-alkoxy-C₁-C₆-alkyl or C₁-C₆-alkoxy, where hydrogen is particularly preferred.

Preferably, both R¹ and R² are C₁-C₄-alkyl or C₁-C₄-alkoxy-C₁-C₄-alkyl, it being particularly preferred for one of these groups to be methyl. Particular preferably, both R¹ and R² are C₁-C₄-alkyl. In particular, one of the radicals R¹ or R² is methyl and the other is C₁-C₄-alkyl, e.g. ethyl.

R³ to R⁹ are preferably H.

Het⁺ is preferably monocyclic. Accordingly, Het⁺ is preferably selected from the compounds of the formulae Het. 1 to Het. 13. Het⁺ is particularly preferably a monocyclic five-membered ring. Accordingly, Het⁺ is particularly preferably selected from the compounds of the formulae Het. 5 to Het. 11 and Het. 13.

Het⁺ is more preferably an imidazolium ion of the formula Het. 5, a pyrazolium ion of the formula Het. 6, an oxazolium ion of the formula Het. 7, a 1,2,3-triazolium ion of the formulae Het. 8 or Het. 9, a 1,2,4-triazolium ion of the formula Het. 10 or a thiazolium ion of the formula Het. 11, where R¹ to R⁵ are as defined above. The statements made above about preferred radicals R¹ to R⁵ apply here correspondingly, i.e. both R¹ and R² are preferably C₁-C₄-alkyl or C₁-C₄-alkoxy-C₁-C₄-alkyl and particularly preferably C₁-C₄-alkyl, it being particularly preferred for one of these groups to be methyl. In particular, one of the radicals R¹ or R² is methyl and the other is C₁-C₄-alkyl, e.g. ethyl. R³ to R⁵ are preferably H.

Het⁺ is even more preferably an imidazolium ion of the formula Het. 5, where R¹ to R⁵ are as defined above. The statements made above about preferred radicals R¹ to R⁵ apply here correspondingly, i.e. both R¹ and R² are preferably C₁-C₄-alkyl or C₁-C₄-alkoxy-C₁-C₄-alkyl and particularly preferably C₁-C₄-alkyl, it being particularly preferred for one of these groups to be methyl. R³ to R⁵ are preferably H. Accordingly, Het⁺ is in particular an imidazolium ion of the formula Het. 5 which has a methyl group on one ring nitrogen atom and a C₁-C₄-alkyl group or C₁-C₄-alkoxy-C₁-C₄-alkyl group on the second ring nitrogen atom. In this case, R³, R⁴ and R⁵ are specifically H. In particular, one of the radicals R¹ or R² is methyl and the other is C₁-C₄-alkyl, e.g. ethyl.

A^{x-}_{1/x} is preferably selected from coordinating anions, i.e. those capable in principle of coordination, e.g. to a metal center.

A^{x-}_{1/x} is preferably selected from halides, pseudohalides, perchlorate, the acid anions of C₁-C₆ monocarboxylic acids and the monoanions and dianions of C₂-C₆ dicarboxylic acids, it being possible for the monocarboxylic acids and dicarboxylic acids to be substituted once, twice or three times by halogen and/or hydroxy. A preferred acid anion is acetate.

A^{x-}_{1/x} is particularly preferably selected from halides, pseudohalides and acetate.

Examples of pseudohalides are cyanide (CN⁻), cyanate (OCN⁻), isocyanate (CNO⁻), thiocyanate (SCN⁻), isothiocyanate (NCS⁻) and azide (N₃⁻).

A^{x-}_{1/x} is in particular chloride, bromide, cyanate, thiocyanate or acetate. A^{x-}_{1/x} is specifically chloride or acetate.

Specifically, Het⁺A^{x-}_{1/x} is an imidazolium chloride Het. 5-Cl⁻ or an imidazolium acetate Het. 5-(CH₃COO⁻), where the imidazolium ion is preferably substituted as described above.

In an alternatively preferred embodiment, the chaotropic liquid is selected from solutions of chaotropic salts in polar aprotic solvents.

The inorganic salts are preferably selected from alkali metal halides, alkaline earth metal halides, ammonium halides, alkali metal pseudohalides, alkaline earth metal pseudohalides, ammonium pseudohalides, alkali metal perchlorates, alkaline earth metal perchlorates and ammonium perchlorates, and mixtures thereof.

The inorganic salt is particularly preferably selected from lithium chloride, calcium thiocyanate, sodium iodide, sodium perchlorate and mixtures thereof.

Preferred polar aprotic solvents are dimethylformamide, dimethylacetamide, dimethyl sulfoxide and diethylamine, and mixtures thereof.

The chaotropic liquid is particularly preferably selected from the ionic liquids described above. Reference is hereby made to the statements made above about the preferred embodiments of the ionic liquid.

Step (i) of the process of the invention is generally carried out in such a way that the polymer which has previously undergone milling if appropriate is mechanically mixed with the chaotropic liquid and stirred until dissolution is complete. In a particular embodiment of the invention, the mixture is heated during or after the mixing to expedite the dissolution and homogenization steps, e.g. by microwave irradiation, but preferably not to a temperature exceeding 150° C., more preferably not exceeding 120° C., in particular not exceeding 100° C.

In a preferred embodiment, the concentration of the solubilized polymer in the chaotropic liquid is 5% by weight to 35% by weight, preferably 5% by weight to 25% by weight and in particular 10% by weight to 25% by weight.

When the solubilized polymer is introduced into the liquid medium (step (ii)) it precipitates within a very short time, e.g. in less than 1 s. The introduction takes place by extrusion, i.e. ejection of the solubilizate through a needle. On introduction of the chaotropic solution into the liquid medium in which the chaotropic components are soluble but the polymer material is substantially insoluble, the polymer precipitates.

Extrusion of the polymer into a liquid medium takes place by means of a movable needle which is preferably a component of an automated apparatus. The needle or the container in which the liquid medium is present, or both, are moved during this in such a way that the extrudate in variant (ii-a) assumes the shape of a three-dimensional scaffold, network or lattice, and in variant (ii-b) assumes the shape of a straight, curved or bent polymer strand.

The liquid medium employed in step (ii) is on the one hand miscible with the chaotropic liquid from step (i) but, on the other hand, the polymer employed is substantially insoluble therein. Preferred liquid media are protic solvents such as water and alkanols, cyclic ethers such as tetrahydrofuran and dioxane, ketones such as acetone and ethyl methyl ketone, and nitriles such as acetonitrile, and mixtures thereof. Preferred liquid media are protic solvents such as water and alkanols, and mixtures thereof. Suitable alkanols are C₁-C₄-alkanols such as, for example, methanol, ethanol, propanol, isopropanol, n-butanol, isobutanol and tert-butanol. The liquid medium is preferably aqueous, i.e. it comprises at least 10% by weight of water. The liquid medium particularly preferably comprises at least 50% by weight of water, especially at least 80% by weight of water. The other constituent of the aqueous medium is preferably selected from C₁-C₃ alkanols such as methanol, ethanol, n-propanol and isopropanol. Water is specifically used.

In a preferred embodiment of the invention, the points of contact or overlap between various elements of the scaffold, lattice or network are stabilized by polyelectrolytes.

Polymers referred to as “polyelectrolytes” are those whose repeating units have a group able to receive or release protons, and which are thus able in a protic, in particular aqueous medium to receive charges and release them again, it being possible for them to be positive and/or negative within a molecule. An example of a group which is negatively charged in aqueous medium is the carboxyl group, and an example of a group which is positively charged in aqueous medium is the amino group. All conventional polyelectrolytes are suitable in principle. Examples of suitable polyelectrolytes are compounds which are employed as additives to increase the wet strength of paper in the manufacture of paper, such as polycarboxylic acids, e.g. polyacrylic acid, polyamines, e.g. polyvinylamine, Polyimines, copolymers of carboxamides unsaturated in the amide moiety and unsaturated carboxylic acids, e.g. N-vinylformamide/acrylic acid copolymers, polymerizable basic heterocycles, e.g. N-vinylpyrrolidone, products of the reaction of polyamines with epichlorohydrin, epoxidized polyamides, urea resins, melamine resins, polyurethanes and the like. Such wet strength agents are described for example in EP 01 118 439, which is incorporated herein by reference.

However, preferred polyelectrolytes are polycarboxylic acids such as, for example, polyacrylic acid, monotonic aliphatic polyamines such as, for example, polyvinylamine, and polymerizable basic heterocycles, i.e. heterocycles having an exocyclic ethylenic double bond such as, for example, polyvinylpyrrolidone.

In a preferred embodiment of the invention, the polyelectrolytes are a constituent of the liquid medium into which the solubilized polymer is extruded. It is preferred in this connection for the liquid medium to comprise up to 20% by weight of polyelectrolytes, in particular from 5% by weight to 10% by weight, based on the total weight of the liquid medium.

The points of contact or overlap between different elements of the scaffold, lattice or network may, however, also be stabilized as described below concerning process variant (ii-b).

In a preferred embodiment of the invention, individual parts or all parts of the scaffold are provided with signaling factors or growth factors which act on living cells. Corresponding factors, e.g. VEGF or NGF, are familiar to the skilled worker. Depending on their individual physical or chemical properties, these factors can preferably either be added to the material to be extruded in step (i) as described above (“doping”) or be coated onto the surface of the resulting strand during the extrusion process in step (ii) or thereafter using a suitable needle.

The resulting polymer scaffold may be homogeneous in relation to the distribution of the factors, but a heterogeneous distribution leading to the formation of signaling factor gradients and thus specifying an orientation for the formation of new tissue, e.g. the ingrowth of blood vessels and nerve fibers is preferred. It is particularly preferred for the heterogeneity of the signaling factors to be combined in a suitable manner with a structural heterogeneity of the scaffold, lattice or network by leaving relatively large, highly doped recesses free e.g. for blood vessels or nerves, or creating the preconditions for the formation of more complex organ structures.

In a preferred embodiment of step (ii-a), the extrusion takes place in such a way that the scaffold is constructed substantially in layers so that a scaffold with a layer structure is formed, i.e. the majority of the rods or strands lies in planes which are parallel to one another, with the stabilizing contacts between the respective adjacent layers being brought about mainly by overlaps of strands, while the contribution of rods or strands not lying in a plane is insignificant for interactions between the layers and thus for the three-dimensional stability of the scaffold. “Constructed substantially in layers” means that the scaffold may also comprise strand arrangements which do not belong to these layers, but the scaffold is constructed mainly, e.g. at least 60%, preferably at least 80% and in particular at least 90%, based on the length of the polymer strands from which the scaffold is overall constructed, from polymer strands arranged in layers.

Each layer preferably consists mainly of extrudate strands running parallel to one another, in particular of bustrophedonic strands, i.e. extrudate strands running alternatingly in one direction and the opposite direction from adjacent strand to adjacent strand. The proportion of elements not running in parallel is insignificant in this case. Stabilization of the strands of each individual layer is primarily achieved through the contact with the strands of the adjacent layer or adjacent layers, in case a lattice-like, permeable structure is to be provided in the relevant region of the plane. However, planes or parts of planes may also have an impermeable configuration through the strands running in parallel in the relevant region being placed so close to one another that they are in contact.

In this connection for mechanical stabilization of the scaffold it is preferred for the strands of adjacent planes to be neither parallel nor antiparallel in relation to one another. It is particularly preferred for the angle of the strands between adjacent layers to be 90°, 60° or 45°.

In a further preferred embodiment of step (ii-a), the extrusion takes place in such a way that the layers are constructed essentially from extrudate strands in the form of two-dimensional space-filling curves (FASS curves; FASS=space-filling, self-avoiding, simple and self-similar). “Essentially” means that at least 60%, preferably at least 80% and in particular at least 90% of the layers, based on the total length of the polymer strands from which the respective layers are constructed, are constructed from polymer strands in the form of two-dimensional space-filling curves. An FASS curve is a path which leads over an area which is composed of a number of uniform fields, or through a three-dimensional or multidimensional space composed of a number of “chambers”, so that each field or each chamber is touched without the path intersecting itself. This results in a structure which is more uniform in both dimensions of the plane than when the plane is composed of parallel strands. Preferred special types of FASS curves are Peano curves, Hilbert curves and Sierpiński curves.

In this embodiment, for production preferably each plane is divided up into a number of areas, each of which is filled out substantially independently of the other areas of the same plane. It is preferred in this connection for adjacent planes to be divided up into different area groups. Each plane is particularly preferably divided up to result in a maximum proportion of square areas which are in each case filled out by a FASS curve. It is particularly preferred in this connection for the areas to be divided up to enable extrusion to be as continuous as possible.

It is likewise particularly preferred in this connection for the plane to be divided up in such a way that it consists substantially, e.g. at least 50%, preferably at least 75%, in particular at least 90%, of FASS curves.

FASS curves can be generated by means of recursive algorithms for a given field. Corresponding methods are familiar to the skilled worker and are described for example in V. Batagelj: Logo to PostScript. Paper prepared for Eurologo'97, Ljubljana 1997; A. J. Cole: A note on space filling curves. Software—Practice and Experience, 13 (1983), 1181-1189; A. J. Cole: A note on Peano Polygons and Gray Codes. International Journal of Computer Mathematics, 18 (1985), 3-13; C. Davis, D. E. Knuth: Number Representations and Dragon Curves, I-II. Journal of Recreational Mathematics, 3 (1970), 66-81; 3 (1970), 133-149; F. M. Dekking, M. Mendes France, A. van der Poorten: Folds !. The Mathematical Intelligencer, 4 (1982), 130-138; 4 (1982), 173-181; 4 (1982), 190-195; F. M. Dekking: Recurrent Sets. Advances in Mathematics, 44 (1982), 78-104; A. J. Fisher: A new algorithm for generating Hilbert curves. Software—Practice and Experience, 16 (1986), 5-12; W. J. Gilbert: Fractal Geometry Derived from Complex Bases. The Mathematical Intelligencer, 4 (1982), 78-86; J. Giles, Jr.: Construction of Replicating Superfigures. Journal of Combinatorial Theory, Series A, 26 (1979), 328-334; L. M. Goldschlager: Short algorithms for space-filling curves. Software—Practice and Experience, 11 (1981), 99; A. Null: Space-filling curves, or how to waste time with a plotter. Software—Practice and Experience, 1(1971), 403-410; P. Prusinkiewicz, A. Lindenmayer: The algorithmic beauty of plants. Springer, New York, 1990; N. Wirth: Algorithms+Data Structures=Programs. Prentice-Hall, 1976; I. H. Witten und B. Wyvill: On the generation and use of space-filling curves. Software—Practice and Experience, 13 (1983), 519-525, which are incorporated herein by reference.

In a further preferred embodiment, the polymer scaffold comprises helical, spiral or circular elements, it being possible for these to be round or angular, continuous or stepwise, single helices or multiple helices. In a preferred embodiment, the helical, spiral or circular elements are different in terms of strand thickness and/or doping from the elements in lattice form.

In a further preferred embodiment, the structure of the polymer scaffold is essentially three-dimensionally homogeneous, i.e. the strands or rods make quantitatively and qualitatively comparable contributions in all spatial dimensions.

In a particularly preferred embodiment of step (ii-a), the polymer scaffold consists essentially of an extrudate strand in the form of a three-dimensional FASS curve and in particular of a three-dimensional Peano curve. “Essentially” means that at least 60%, preferably at least 80% and in particular at least 90% of the scaffold, based on the total length of the polymer strands from which the scaffold is overall constructed, is constructed from polymer strands in the form of three-dimensional FASS curves.

In a further preferred embodiment, at least 25% of the total volume of the polymer scaffold is occupied by continuous channels. A “continuous channel” is a cavity whose length is at least half the length of the dimension, parallel thereto, of the complete polymer scaffold, and which communicates with the outer surface of the scaffold.

The polymer scaffold is isolated either by taking it out of the container used in step (ii) or by first removing the liquid medium into which extrusion has taken place. An alternative isolation method, which is suitable in particular on use of water or aqueous mixtures as liquid medium in step (ii), is to freeze the medium and isolate the scaffold from the frozen medium by suitable methods, i.e. by mechanical removal of the frozen medium or by sublimation thereof. The scaffold can then be freed of residues of the liquid medium, e.g. by drying in air, in a drying oven or a vacuum oven or by lyophilizatiion.

In variant (ii-b), the solubilizate obtained in step (i) is extruded in such a way that individual straight, curved or bent polymer strands are produced. The desired shape is produced by the relative movement of needle to container and/or by shaping the strand after the extrusion, e.g. by stretching, curving and/or bending. It is possible to use for this purpose all conventional mechanical aids such as clamps, tweezers, rods, etc. or else molds which have the desired shape and are immersed in the liquid medium and subsequently removed again.

Before being processed to form the scaffold, the polymer strand is preferably isolated from the liquid medium, if appropriate (post-)formed and/or dried. The isolation and drying can be carried out as previously described. The (post-)forming can take place after or, preferably, before the drying. The (post-)forming may comprise for example stretching, curving and/or bending the polymer strand, e.g. with the aid of the aforementioned aids.

The polymer strands (fibers) can then be linked to give the desired scaffold structure. It is possible in this connection to link together either only polymer fibers of the same type or different polymer fibers. When different polymer fibers are used, they may differ for example in their diameter, in their nature and/or in their production process. It is thus possible to use polymer fibers which differ in that they have been produced by extrusion with needles differing in shape and/or differing in diameter, and/or in that they have been produced starting from different biodegradable and biocompatible polymers and/or in that they have been produced by different processes, it being necessary for at least one type of polymer strand to have been produced by the process of the invention. Processes which differ from the process of the invention and which can be employed are all processes familiar to the skilled worker and suitable for the particular type of polymer for producing polymer fibers, such as spinning processes, electro-spinning, etc.

The linkage can take place by means of known techniques for joining/bonding polymers of these types, e.g. by means of biodegradable and biocompatible adhesives customary for this purpose. However, the linkage preferably takes place by applying a small amount of the solubilizate obtained in step (i) or of another one composed of a biocompatible and biodegradable polymer in a chaotropic liquid to the desired linkage points, and subsequently adding a liquid medium in which the polymer is insoluble. When the polymer precipitates it simultaneously joins the individual polymer strands together.

It is also possible in principle to link the polymer strands together in liquid medium, for example by applying a small amount of the solubilizate obtained in step (i) to the desired linkage points. The scaffolds produced in liquid medium can than be isolated as described above and dried if desired. The first procedure, i.e. initial isolation of the polymer strands and only subsequently linkage to give a scaffold is, however, preferred because it is easier to carry out.

There is often, especially when only small amounts of the liquid medium are used, formation in step (ii) of gelatinous products which can be isolated relatively easily. Conversion into the solid state takes place by drying. It may also be beneficial to leave the scaffolds formed in gelatinous form until they are employed, in order to increase their storability, and to dry them only shortly before use thereof.

If the scaffolds are not yet (completely) dry, they can if desired be (post-)formed, which can take place as described previously.

Variant (ii-a) is preferred for step (ii) in particular for producing scaffolds of complex shape. This variant allows in particular simple and reproducible access to three-dimensional scaffolds whose production would otherwise not be trivial. However, a procedure according to variant (ii-b) is also suitable for producing simple, especially two-dimensional, scaffolds, e.g. nets as are sufficient for example for constructing flat tissue such as skin.

The resulting scaffolds can then be treated as described above hereinafter, e.g. by coating or doping with signaling and/or growth factors which act on living cells or by colonization with living cells.

It is possible by the process of the invention easily to produce two- and three-dimensional scaffolds from biodegradable and biocompatible polymers which can ordinarily be processed only with difficulty, such as cellulose or cellulose derivatives. These scaffolds, which can also assume highly complex shapes, can be employed as shaping structures in the construction of artificial tissue.

The present invention further relates to a polymer scaffold which is obtainable by the process of the invention. Concerning preferred embodiments of the polymer scaffold, reference is also made to the statements above.

In a preferred embodiment, a “negative scaffold” is formed by casting another polymer which can be melted or gelled, and which differs in degradability in vitro from the first polymer, in the voids of the finished primary scaffold, followed by degradation of the first polymer.

In a preferred embodiment of the polymer scaffold of the invention, living cells are bound thereto. These are preferably eukaryotic cells, in particular mammalian cells, e.g. human cells. Alternatively, the living cells are preferably prokaryotic cells, in particular cells of socially organized bacteria, e.g. bacteria which form biofilms or grow in mycelia.

Before the polymer scaffold is colonized by living cells, it can be prepared in a suitable manner. The preparation of the finished polymer scaffold for colonization by living cells can thus take place for example by washing one or more times with an aqueous medium, e.g. water, physiological saline (“Ringer's solution”) or phosphate-buffered physiological saline (PBS). Multiple washings are appropriate especially when the polymer used and/or the polyelectrolytes used comprise a significant proportion of low molecular weight substances.

Furthermore, the polymer scaffold can be dried before the colonization by living cells, e.g. by rapid freezing followed by freeze drying. It is preferred in this connection for the drying parameters to be chosen so that the dried polymer scaffold is storable. In this connection, storability means that the polymer scaffold shows no damage evident under the light or electron microscope to the structure in a period of, preferably, at least one week, particularly preferably at least one month.

The dried scaffold is preferably equilibrated with an aqueous medium before the colonization by living cells, it being possible for the equilibration step to be designed as washing step or to be followed by one or more washing steps. The equilibration may in addition comprise initially impregnating the polymer scaffold with substances which bind specifically or nonspecifically to the surface of the polymer strands. Such an impregnation can also take place without previous drying steps. Preferred molecules for an impregnation are those which modulate or influence the colonization and/or function of the living cells, but are not compatible with the extrusion process of the invention, e.g. because of lack of stability in relation to the chaotropic substances used. It is preferred in this connection for either the desired distribution of the substance to be absorbed on the polymer strands to be substantially homogeneous or for the various polymer strands to be designed so that they have a different affinity for the substance to be absorbed, so that a differential distribution of the substance to be absorbed results.

The equilibration/impregnation is suitably carried out especially when molecules bound to the surface of the polymer strands are to be activated, e.g. by elimination of protective groups, activating proteolytic cleavage of proenzymes and/or renaturation of polypeptide chains which have been denatured as a result of the treatment with chaotropic substances, for example by treating the scaffold with proteins or protein mixtures having chaperone activity under weakly reducing conditions, e.g. by incubation with a physiological saline buffer comprising 10% by weight serum albumin and 1 mM β-mercaptoethanol at +37° C.

Furthermore, the polymer scaffold can be mechanically pretreated before the colonization of the polymer scaffold by living cells, e.g. by stretching or pretensioning. Such processes are familiar from polymer technology; it is assumed, without being bound by the theory, that the application of small mechanical forces to a polymer strand leads to an improvement in the supermolecular arrangement and thus to enhancement of the intermolecular interactions and an increase in the mechanical stability of the strand.

If desired, further mechanical, chemical, thermal and radiation treatments of the polymer scaffold are possible before the colonization by living cells.

The colonization of the prepared polymer scaffold by living cells in principle takes place in vitro, while degradation of the polymer scaffold, formation of extracellular matrix etc may if appropriate continue after the implantation. The cells primarily used for the colonization are adherent or capable of adhesion and have previously been detached from their natural assemblage, e.g. by treatment with proteases, preferably trypsin, and/or chelating agents such as, for example, ethylenediaminetetraacetic acid (EDTA). Corresponding processes for extracting cells from their assemblages are familiar to the skilled worker.

The colonization expediently takes place by incubating the prepared polymer scaffold, which has been equilibrated with the cell growth medium if appropriate, with the cells in a growth medium under generally permissive conditions. Typical conditions for the colonization by human cells are, for example: DMEM (Dulbecco's modification of Eagle's medium) supplemented with 10% fetal calf serum and suitable antibiotics, at +37° C. under an atmosphere with 5% CO₂. Such media and conditions are familiar to the skilled worker. The colonization and construction of tissue can be monitored in various ways, e.g. in situ by light microscopy. Use may be preceded by further washings and an adjustment of the medium to more body-like conditions.

Preformed structures suitable for biochemically/physiologically active tissues must have a three-dimensional fine structure which makes colonization by cells possible in vitro, allows these cells to be supplied adequately with oxygen and nutrients, and later permits the ingrowth of blood vessels (vascularization) and, if appropriate, nerves from the organism. For this purpose and in order to construct an organ or organ part of complex structure (e.g. a nephron), it is also desirable to be able to “dope” individual parts of the preformed synthetic structure with suitable growth factors and signaling factors in order in this way to organize the self-organization of the cells to functional assemblages. Growth factors which stimulate for example the ingrowth of blood vessels or nerves into a tissue region, and signal substances important for establishing and maintaining structures in the organism, are at least in principle familiar to the skilled worker. A review is given for example by Bukovsky, “Cell-mediated and neural control of morphostasis”, Med. Hypotheses 36 (1991), 261-268.

The invention further relates to the use of a polymer scaffold to which living cells are bound as described above for producing an implant for restoring, measuring or modifying biological functions in the organism to be treated. In a preferred embodiment, the implant is selected from artificial bone tissue, artificial skin, artificial blood vessels and hollow organs. In an alternatively preferred embodiment, the implant serves as carrier in a drug delivery system or an implantable slow-release formulation.

The invention further relates to an artificial tissue which is constructed on a polymer scaffold of the invention. In this case, the polymer scaffold may at the time of implantation or other use still be substantially completely retained, partially degraded and/or replaced by extracellular matrix or be substantially completely degraded and/or replaced by extracellular matrix.

In a preferred embodiment, the implant serves as nerve guide for restoring broken nerve fibers. It is preferred in this connection for the polymer scaffold not yet to be completely degraded at the time of implantation.

In an alternatively preferred embodiment, the tissue is selected from artificial bone tissue, artificial skin, artificial blood vessels and hollow organs. If the tissue is an artificial blood vessel or hollow organ, it preferably comprises helical elements, because these have a geometry suitable for producing the inner and outer surface.

The present invention further relates to the use of an artificial tissue based on a polymer scaffold of the invention for diagnostics ex vivo and in vitro.

The invention further relates to the use of a polymer scaffold of the invention, in which living cells have become bound to the scaffold, in a bioreactor. In a particular embodiment, cells are in this case kept under steady state conditions, e.g. by use of a countercurrent exchange. It is preferred in this connection for the cells to secrete soluble products, and hybridomas or stable transfectants which form a soluble protein are particularly preferred. In this embodiment, three-dimensional polymer scaffolds form a more robust alternative to the hollow fiber systems known in the art (see, for example, T. L. Evans and R. A. Miller, “Large-scale production of murine monoclonal antibodies using hollow fiber bioreactors”, Biotechniques 1988, Sep. 6 (8): 762-767).

If eukaryotic cells are used according to the invention in a bioreactor, hybridomas and other antibody-producing cells, e.g. quadromas, are preferred. Stably transfected cells, e.g. CHO or NIH3T3 cells, e.g. having a transgene integrated into the genome, are likewise preferred, it being particularly preferred for the cells to secrete a soluble protein.

If prokaryotic cells are used according to the invention in a bioreactor, socially organized producers of low molecular weight metabolites are preferred, in particular producers of antibiotics, e.g. streptomycetes, e.g. Streptomyces caelicolor.

The present invention further relates to an apparatus for carrying out the process of the invention, in particular for producing a polymer scaffold by extrusion. In a preferred embodiment, the apparatus comprises an extrusion needle which is movable in three dimensions relative to the resulting lattice, a mechanical positioner and a computer unit suitable for controlling the positioner. It is particularly preferred for the computer unit to comprise a program for automatic generation of the structures.

In a particular embodiment, the extrusion mechanism and/or the scaffold formed to date can be rotated around a fixed or variable axis.

Corresponding mechanical apparatuses for relative three-dimensional positioning of the needle are familiar in principle to the skilled worker (see, for example, T. H. Ang et al., “Fabrication of 3D chitosan-hydroxyapatite scaffolds using a robotic dispensing system”, Materials Science and Engineering C 20 (2000): 35-42), as are the principles of the extrusion of polymers.

In a particular embodiment, the three-dimensional movement mainly takes place in steps running parallel to the three axes of the resulting scaffold. It is preferred in this connection for the needle to be held parallel to none of the three axes, and in particular for it to form a maximum angle with all three axes (arc tan √2≈55°; corresponds to the point 111 in the Cartesian coordinate system).

In a further preferred embodiment, the needle is parallel to one of the axes of the scaffold. It is preferred in this connection for the scaffold to be movable in one dimension and the extrusion mechanism in the two others, and for the needle to be held parallel to the movement dimension of the scaffold (“Z axis”).

In a particular embodiment, the cross section of the needle is round. In another particular embodiment, the needle has an oval, polygonal, serrated or irregularly shaped cross section. In this connection, it is possible in the present invention to employ for producing a scaffold simultaneously or successively a plurality of needles with identical or different diameter and with identical or different cross sectional geometry.

The apparatus of the invention comprises in principle three groups of components:

1. storage container, system of lines and needle for chaotropic solutions,
2. positioning system for the needle,
3. container for the liquid medium.

The first component comprises those parts which come into direct contact with the solution of the polymer in a chaotropic solvent. The corresponding parts are therefore expediently made of materials which are resistant to the chaotropic agents used, which have inter alia a corrosive effect. With a view to the later use of the finished scaffolds, it is preferred for it to be possible to manipulate the parts of the first component aseptically, e.g. by them being sterilizable by superheated steam (“autoclavable”).

The storage container preferably consists of silicate materials or corrosion-resistant metal, e.g. glass, ceramic or stainless steel. The needle preferably consists of corrosion-resistant metal, e.g. stainless steel. The line leading from the storage container to the movable needle usually comprises parts which are flexible and, in a particular embodiment, also rigid. The flexible parts of the line are suitably made from corrosion-resistant polymer material, e.g. silicone. If rigid parts are used as elements of the line, these can in principle be made from the same materials as the storage container or from the same materials as the flexible parts.

The storage container is used to receive the polymer dissolved in a chaotropic agent of the invention. In a particular embodiment of the invention, it is equipped with a stirrer in order to ensure the homogeneity of the polymer solution. In a further particular embodiment, the storage container is temperature-controlled, it being preferred for it to be possible to keep the contents of the storage container at a temperature at which the solution of polymer in chaotropic solution is liquid. Both stirrer and temperature-control unit can be connected independently of one another to the control system of the second component, or be independent thereof.

The storage container can in principle have any suitable shape.

The dissolved polymer is removed from the storage container through the line connected to the storage container, either following gravity, e.g. with a valve control, or, preferably, through a controlled pump. In this case, preferred pump mechanisms require no direct contact of movable constituents with the solution to be pumped, e.g. peristaltic tube pumps. It is preferred in this connection for the parts (valves and/or pumps) serving to control the flow rate to be connected to the control system of the second component.

The second component comprises mechanical components and, preferably, software to control the relative movement between needle and extrudate. The mechanical components are known in principle in this connection. In a preferred embodiment, the second component group comprises a number of stepping motors which actuate fine drives which are disposed perpendicular to one another and which serve to shift the needle. In this connection, the second component preferably comprises as many stepping motors/fine drives as the needle has degrees of freedom of lateral movement.

In a particular embodiment of the invention, the second component group additionally comprises an apparatus for changing the angle of the needle. In a further particular embodiment, the second component group comprises an apparatus for rotating the needle. In a further particular embodiment, the second component group comprises an apparatus for automatically changing between different needles of different diameter and/or different geometry, e.g. on the revolver principle.

In a preferred embodiment of the invention, the stepping motors and optional components of the second group are controlled by a computer by means of a D/A converter. It is particularly preferred for the valves and pumps of the first group and, in particular, also the stirrer and temperature-control unit of the first group also to be controlled correspondingly.

In a preferred embodiment, the computer uses commercially available hardware, and software suitable for three-dimensional control of the needle. It is particularly preferred in this connection for the software to be able to convert a predefined spatial shape automatically into an arrangement according to the invention of extrudate strands, in particular an arrangement comprising FASS curves, specifically Peano curves, of extrudate strands, and to guide the needle correspondingly. In a particular embodiment of the invention, the controlling computer also controls the pump which belongs to the first component group and which controls the supply of dissolved polymer to the needle, appropriate for the movement of the needle.

Corresponding stepping motors, fine drive systems, D/A converters and suitable computer hardware components are familiar in principle to the skilled worker.

The third component group comprises the container for the liquid medium in which the extrusion of the dissolved polymer material takes place. Any type of container is suitable in principle for this purpose. In a particular embodiment of the invention, the water tank consists of glass or ceramic. In a preferred embodiment of the invention, the water tank is likewise supported on a system of stepping motors and fine drives which are able to add the missing degrees of freedom of the needle in lateral displacement and/or rotation. It is expedient for the stepping motors and fine drives of the third component group to be controlled by the same hardware and software as those of the second, so that uniform control of all the movements within the system is possible for precise shaping.

With a view to the later use of the finished scaffolds, it is preferred for it to be possible also to manipulate the parts of the third component aseptically, e.g. by them being sterilizable by superheated steam (“autoclavable”).

The invention is illustrated by the following, non-limiting example and the FIGURE.

The FIGURE shows a cellulose net which has been produced as in the example from two directions of view. The depicted 1 cent coin illustrates the size of the net.

### EXAMPLE

Cellulose was introduced into 1-ethyl-3-methylimidazolium acetate and dissolved therein by stirring at 90° C. for two hours. The cellulose content of the solution was 1% by weight cellulose based on the total weight of the solution.

This solution was injected by means of a surgical needle into a water bath at a rate of 70 ml/h and with stretching of the resulting polymer fibers. The result was a gel which shrank on drying and formed free fibers. The dry fibers had on average a diameter of 70 μm. Laying the gels one on top of the other and linking the joining points between the fibers by applying one drop of the cellulose solution prepared above and then one drop of water resulted in net-like structures (see FIGURE).

1. A process for producing two- or three-dimensional scaffolds of biodegradable and biocompatible polymers which comprises the following steps:
(i) solubilization of a biodegradable and biocompatible polymer in a chaotropic liquid; and
(ii-a) substantially continuous extrusion of the solution obtained in step (i) into a liquid medium which is miscible with the chaotropic liquid but in which the polymer is substantially insoluble, by means of a needle, where the needle and the resulting scaffold move relative to one another during the extrusion; or
(ii-b) extrusion of the solution obtained in the first step into a liquid medium which is miscible with the chaotropic liquid but in which the polymer is substantially insoluble, by means of a needle to form individual straight, curved or bent polymer strands, where the needle and the resulting polymer strand move relative to one another during the extrusion step, if appropriate isolation of the polymer strands from the liquid medium and linkage of the polymer strands to form a two- or three-dimensional scaffold.

2. The process according to claim 1, where the polymer is a polysaccharide or modified polysaccharide.

3. The process according to claim 2, where the polysaccharide is cellulose or a cellulose derivative.

4. The process according to claim 1, where the chaotropic liquid has a melting point of less than or equal to 150° C.

5. The process according to claim 1, where the chaotropic liquid is selected from salts of the formula Het⁺A^{x-}_{1/x}, in which
Het⁺ is a positively charged N-alkylated, N-arylated, N-arylalkylated, N-alkoxylated, N-aryloxylated, N-arylalkoxylated, N-alkoxyalkylated and/or N-aryloxyalkylated nitrogen-containing heterocycle;
A^{x-}_{1/x} is an anion; and
x is 1, 2 or 3.

6. The process according to claim 5, where Het⁺ is selected from
positively charged 5- or 6-membered aromatic heterocycles which comprise as ring member a group NR^{a} and optionally one to three heteroatoms or heteroatom-containing groups which are selected from N, O, S, NR^{b}, SO and SO₂,
positively charged 5- or 6-membered aromatic heterocycles which comprise as ring member a group NR^{a} and optionally one or two heteroatoms or heteroatom-containing groups which are selected from N, O, S, NR^{b}, SO and SO₂, and which are fused to a benzene ring, and
positively charged 5- or 6-membered saturated alicyclic heterocycles which comprise as ring member a group NR^{a}R^{a′} and optionally one or two heteroatoms or heteroatom-containing groups which are selected from O, S, NR^{b}, SO and SO₂,
in which
R^{a} and R^{a′} are independently of one another C₁-C₆-alkyl, aryl, C₁-C₆-alkoxy, aryloxy, C₁-C₆-alkoxy-C₁-C₆-alkyl or aryloxy-C₁-C₆-alkyl; and
R^{b} is hydrogen, C₁-C₆-alkyl, aryl, C₁-C₆-alkoxy, aryloxy, C₁-C₆-alkoxy-C₁-C₆-alkyl or aryloxy-C₁-C₆-alkyl;
where the alicyclic or aromatic heterocycles or the benzene rings to which the latter may be fused may have 1 to 5 substituents selected from C₁-C₆-alkyl, C₁-C₆-alkoxy and C₁-C₆-alkoxy-C₁-C₆-alkyl.

7. The process according to claim 6, where Het⁺ is selected from compounds of the formulae Het.1 to Het.15 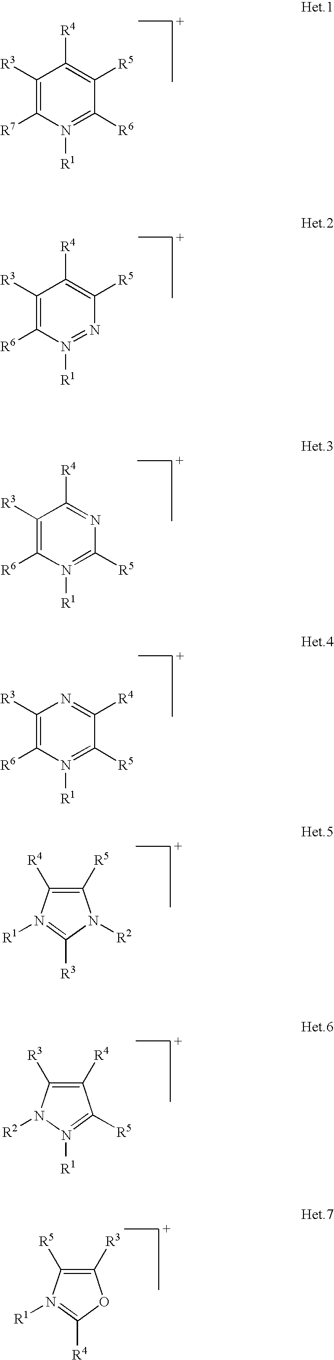 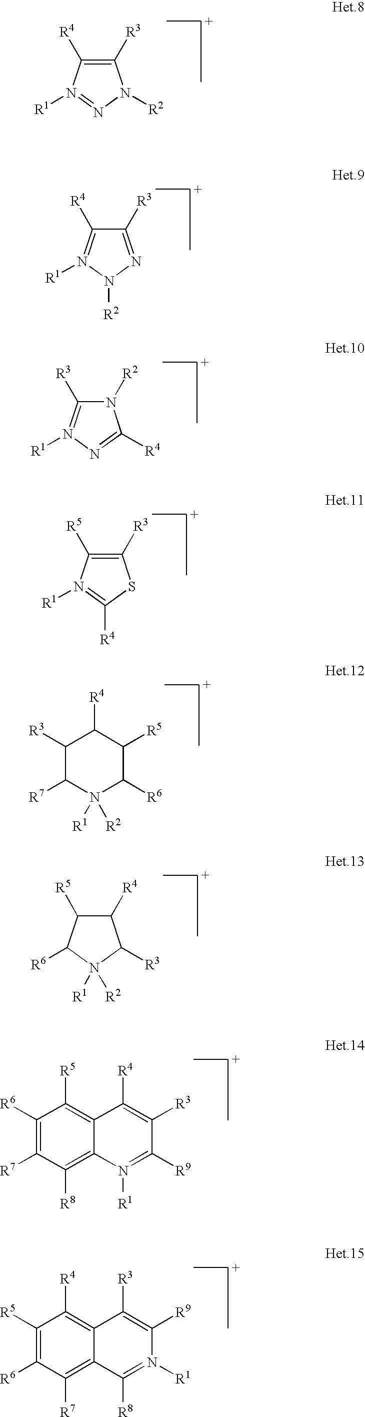
in which
R¹ and R² are independently of one another C₁-C₆-alkyl or C₁-C₆-alkoxy-C₁-C₆-alkyl; and
R³ to R⁹ are independently of one another hydrogen, C₁-C₆-alkyl, C₁-C₆-alkoxy or C₁-C₆-alkoxy-C₁-C₆-alkyl.

8. The process according to claim 7, where Het⁺ is selected from imidazolium ions of the formula Het.5, pyrazolium ions of the formula Het.6, oxazolium ions of the formula Het.7, 1,2,3-triazolium ions of the formulae Het. 8 or Het.9, 1,2,4-triazolium ions of the formula Het.10 and thiazolium ions of the formula Het. 11.

9. The process according to claim 5, where A^{x-}_{1/x} is selected from halides, pseudohalides, perchlorate, the acid anions of C₁-C₆ monocarboxylic acids and the monoanions or dianions of C₂-C₆ dicarboxylic acids, it being possible for the monocarboxylic acids and dicarboxylic acids to be substituted once, twice or three times by halogen and/or hydroxy.

10. The process according to claim 9, where A^{x-}_{1/x} is selected from halides and pseudohalides.

11. The process according to claim 1, where the chaotropic liquid is selected from solutions of inorganic salts in polar aprotic solvents.

12. The process according to claim 11, where the inorganic salts are selected from alkali metal halides, alkaline earth metal halides, ammonium halides, alkali metal pseudohalides, alkaline earth metal pseudohalides, ammonium pseudohalides, alkali metal perchlorates, alkaline earth metal perchlorates, ammonium perchlorates and mixtures thereof.

13. The process according to claim 11, where the polar aprotic solvent is selected from dimethylformamide, dimethylacetamide, dimethyl sulfoxide, diethylamine and mixtures thereof.

14. The process according to claim 1, where the liquid medium employed in step (ii-a) or (ii-b) is aqueous.

15. The process according to claim 1, where the needle is a component of an automated apparatus.

16. The process according to claim 1, where individual parts or all parts of the scaffold are coated or doped with signaling factors or growth factors which act on living cells.

17. The process according to claim 1, where the scaffold in step (ii-a) has a substantially layered structure.

18. The process according to claim 17, where the layers of the scaffold are constructed essentially of extrudate strands running in parallel.

19. The process according to claim 17, where the layers of the scaffold are constructed essentially from extrudate strands in the form of FASS curves.

20. The process according to claim 1, where the scaffold in step (ii-a) is essentially constructed from an extrudate strand in the form of a three-dimensional FASS curve.

21. A polymer scaffold obtainable by a process according to claim 1.

22. The polymer scaffold according to claim 21, which comprises living cells bound to the polymer scaffold.

23. The method of using a polymer scaffold according to claim 21 for producing an implant for restoring, modifying or measuring biological functions.

24. The method of using a polymer scaffold according to claim 21 in a bioreactor.

25. An artificial tissue comprising a polymer scaffold according to claim 21.

26. The method of using an artificial tissue according to claim 25 for ex vivo and in vitro diagnostics.
